(19) United States
(12) Patent Application Publication
(10) Pub. No.: US 2015/0065768 A1
(43) Pub. Date: Mar. 5, 2015

(54) SYSTEMS AND METHODS FOR XYLENE ISOMER PRODUCTION

(71) Applicant: UOP LLC, Des Plaines, IL (US)

(72) Inventors: Leonid Bresler, Northbrook, IL (US); Robert B. Larson, Naperville, IL (US); John B. Robertson, Lake Zurich, IL (US)

(73) Assignee: UOP LLC, Des Plaines, IL (US)

(21) Appl. No.: 14/014,134

(22) Filed: Aug. 29, 2013

(51) Int. Cl. C07C 6/06; C07C 5/27

(52) U.S. Cl. CPC C07C 6/06; C07C 5/2729
USPC 585/304; 585/478

(57) ABSTRACT

Methods and systems are provided for producing a xylene product. The method includes fractionating a feed stream in a feed fractionator to produce a feed bottoms stream and a feed overhead stream. The feed stream includes aromatic compounds and non-aromatic compounds, and more than 5 weight percent of the non-aromatic compounds have a boiling point above 105° C. at one atmosphere of pressure. The feed bottoms stream is de-ethylated in a heavy aromatics conversion zone to produce a de-ethylated aromatics stream and a light gases stream, where non-aromatic compounds are converted to light gases in the light gases stream. The de-ethylated aromatics stream is fractionated to produce a heavy aromatics stream and an intermediate aromatics stream, and a desired isomer stream is recovered from the intermediate aromatics stream and an isomerized stream in an isomer recovery process. The isomer recovery process produces an isomer raffinate stream, and the isomer raffinate stream is isomerized in an isomerization zone to produce the isomerized stream.

### TECHNICAL FIELD

The present disclosure generally relates to systems and methods for producing desired isomers of xylene, and more particularly relates to systems and methods for converting non-reformed hydrocarbon streams into desired isomers of xylene.

### BACKGROUND

Xylene isomers are important intermediates in chemical syntheses, and specific xylene isomers are desired for different processes. Para-xylene is a feedstock for terephthalic acid, and terephthalic acid is used in the manufacture of synthetic fibers and resins. Meta-xylene is used in the manufacture of certain plasticizers, azo dyes, and wood preservatives. Ortho-xylene is a feedstock for phthalic anhydride production, and phthalic anhydride is used in the manufacture of certain plasticizers, dyes, and pharmaceutical products.

Reactions that produce xylene generally produce the xylene isomers in ratios that do not match the demand, and also produce ethyl benzene which is difficult to separate from the xylene. The demand for para-xylene in particular exceeds the production ratios, and several methods have been developed for adjusting the amount of para-xylene recovered from various production processes. The isomer production ratio can be adjusted to meet commercial demand by combining xylene isomer recovery, such as by selective adsorption and/or crystallization, with isomerization to yield additional quantities of the recovered isomer. The xylene isomer recovery changes the ratio of the xylenes to a non-equilibrium value lean in the recovered isomer, and isomerization adjusts the isomer ratio back towards the equilibrium value.

In many xylene isomer recovery processes, aromatics compounds with 9 carbons or more (C9+ aromatics) are present in the feed stream, where xylene is a C8 aromatic. In this description, the abbreviation of “C” followed by a number indicates the number of carbons present in the molecule, and a “+” sign afterwards indicates the indicated number of carbons or more. For example, C7+ means a molecule with 7 carbons or more. A “−” sign after the number indicates the number or less, so C7− means a molecule with 7 carbons or less. The C9+ aromatics are undesirable in the xylene isomer recovery process because they decrease performance, such as by reducing catalyst and/or adsorbent life. Therefore, the feed stream is fractionated to separate the C9+ aromatics, which involves vaporizing and re-condensing, or lifting, the entire C8 aromatics portion. Lifting the entire C8 aromatics portion of the feed stream 10 is expensive because of the high energy demand. Xylene isomer recovery uses an isomer recovery unit and an isomerization unit, where the xylene flows in a loop through the two units. In many existing processes, an isomerized stream flows from the isomerization unit to the isomer recovery unit as part of the loop, and the isomerized stream is fractionated to remove C9+ compounds. The entire C8 aromatics portion is repeatedly lifted as the isomerized stream flows to the isomer recovery unit, and this requires energy that increases the operating costs. Larger equipment is required to lift larger quantities of xylene, so there are also increased capital costs to build and install larger equipment.

The feed stream is reformed before entering existing xylene isomer recovery processes, and a reforming process that produces large quantities of aromatic compounds is used. Other processes may also produce large quantities of aromatic compounds. Declining gasoline demand in many countries can lead to fluid catalytic cracking (FCC) units being operated in high severity mode to increase the production of propylene. FCC units operated in high severity mode also produce higher quantities of aromatic compounds with molecules having about 7 to about 10 carbons (C7-10). The FCC products are fractionated, so a C7-10 stream that is high in aromatics is available without reforming. The reforming process increases the operating costs, increases capital costs for manufacture and installation, and can reduce the total quantity of xylene. There are other processes that produce C7-10 streams that are high in aromatics, such as the production of liquid products from coal, and adding a reforming operation increases operating costs and capital costs in the same manner as for an FCC unit.

Accordingly, it is desirable to develop methods and systems for producing desired xylene isomers from aromatic rich feedstocks that are not reformed. In addition, it is desirable to develop methods and systems for producing desired xylene isomers without reforming the feedstock, where the xylene isomer recovery process is energy efficient. Furthermore, other desirable features and characteristics of the present embodiment will become apparent from the subsequent detailed description and the appended claims, taken in conjunction with the accompanying drawings and this background.

### BRIEF SUMMARY

A method is provided for producing xylene. The method includes fractionating a feed stream in a feed fractionator to produce a feed bottoms stream with aromatic compounds having 8 carbons or more and a feed overhead stream with aromatic compounds having 7 carbons or less. The feed stream includes aromatic compounds and non-aromatic compounds, and more than 5 weight percent of the non-aromatic compounds have a boiling point above 105° C. at one atmosphere of pressure. The feed bottoms stream is de-ethylated in a heavy aromatics conversion zone to produce a de-ethylated aromatics stream and a light gases stream, where non-aromatic compounds are converted to light gases in the light gases stream. The de-ethylated aromatics stream is fractionated to produce a heavy aromatics stream and an intermediate aromatics stream, and a desired isomer stream is recovered from the intermediate aromatics stream and an isomerized stream in an isomer recovery process. The isomer recovery process produces an isomer raffinate stream, and the isomer raffinate stream is isomerized in an isomerization zone to produce the isomerized stream.

Another method is also provided for producing xylene. The method includes fractionating a feed stream in a feed fractionator to produce a feed bottoms stream and a feed overhead stream, where the feed stream has more than 30 weight percent non-aromatic compounds. The compounds in the feed overhead stream are separated into a non-aromatics stream and a first light aromatics stream, where the first light aromatics stream includes toluene. The feed bottoms stream is contacted with a heavy aromatics conversion catalyst to obtain a de-ethylated aromatics stream and a light gases stream. The de-ethylated aromatics stream is fractionated to produce a heavy aromatics stream with aromatic compounds having 9 carbons or more, and an intermediate aromatics stream with aromatic compounds having 8 carbons. The intermediate aromatics stream and an isomerized stream are subjected to an isomer recovery process to produce a desired isomer stream and an isomer raffinate stream. The isomer raffinate stream is contacted with an isomerization catalyst at isomerization conditions to produce the isomerized stream. The heavy aromatics stream and the first light aromatics stream are contacted with a transalkylation catalyst to produce a transalkylation stream.

### BRIEF DESCRIPTION OF THE DRAWINGS

Various embodiments will hereinafter be described in conjunction with the following figures, wherein like numerals denote like elements, and wherein:

FIG. 1 is a schematic diagram of an exemplary embodiment of a fluid catalytic cracking unit that produces a feed stream; and

FIG. 2 is a schematic diagram of an exemplary embodiment of a xylene isomer product system.

### DETAILED DESCRIPTION

The following detailed description is merely exemplary in nature and is not intended to limit the various embodiments and the application and uses thereof. Furthermore, there is no intention to be bound by any theory presented in the preceding technical field, background, brief summary, or the following detailed description.

The various embodiments relate to systems and methods for producing a desired xylene isomer from a hydrocarbon feedstock that has not been reformed. Xylene production is simplified by using a feedstock that has not been reformed. The feed stream is fractionated to produce a feed overhead stream and a feed bottoms stream, and various process streams are removed from the feed overhead stream to leave a first light aromatics stream rich in toluene. The feed bottoms stream is fed to a heavy aromatics conversion zone that de-ethylates heavy aromatics, such as methyl ethyl benzene, and also converts non-aromatic compounds into smaller non-aromatic compounds that are vented in a light gases stream. The de-ethylated stream is fractionated to produce an intermediate aromatics stream rich in xylene, and a heavy aromatics stream rich in C9+ aromatic compounds. The heavy aromatics stream and the light aromatics stream are fed to a transalkylation zone that produces benzene and xylene from toluene and C9+ aromatics. The xylene from the transalkylation zone is fed into the heavy aromatics conversion zone to increase the quantity of xylene. The intermediate aromatics stream is fed into a xylene isomer recovery process to recover the desired xylene isomer.

Aromatic reforming is a process that re-arranges and re-structures hydrocarbon molecules with a propensity to produce aromatic compounds. Aromatic reforming is not 100 percent efficient, so some of the hydrocarbons are broken into smaller molecules or re-arranged to form branched paraffins. There are several different reforming methods and catalysts for different purposes, and one of those methods is catalytic reforming for aromatics. Catalytic reforming is a chemical process that re-arranges or re-structures hydrocarbon molecules, and typically breaks some of the hydrocarbon molecules into smaller molecules. Aromatic reforming is used when aromatic products are desired, but aromatic reforming favors benzene (C6) or toluene (C7) over xylene or ethyl benzene (C8). In some embodiments, aromatic reforming produces about 90 percent C6 and C7 aromatics, so only a small percentage of the resulting product stream is C8 aromatics. In fact, in some embodiments aromatic reforming can actually reduce the amount of C8 aromatics, because the C8 aromatics are converted to other products, such as the C7 or C6 aromatics. In some embodiments, a feed stream is richer in C8 aromatics before reforming, so reforming reduces the total xylene available. Many embodiments of aromatic reforming also reduce the fraction of C8+ non-aromatics, typically to less than 5 mass percent of the total quantity of non-aromatics. Prior to reforming, the C8+ non-aromatics are more than 5 mass percent of the total quantity of non-aromatics, so the reforming process shifts the average molecular weight of the non-aromatics downward. In several embodiments, a hydrocarbon stream has not been reformed if the C8+ non-aromatic compounds are more than about 5 mass percent of the total non-aromatic compounds, and the feed stream has been reformed if it is less than about 5 mass percent of the total non-aromatic compounds. C8+ non-aromatic compounds generally boil at 105° C. or greater at one atmosphere of pressure, and C7-non-aromatic compounds generally boil below 105° C. at one atmosphere of pressure, so a hydrocarbon stream where more than 5 weight percent of the non-aromatic compounds have a boiling point above 105° C. at one atmosphere of pressure generally indicates a feed stream that has not been reformed.

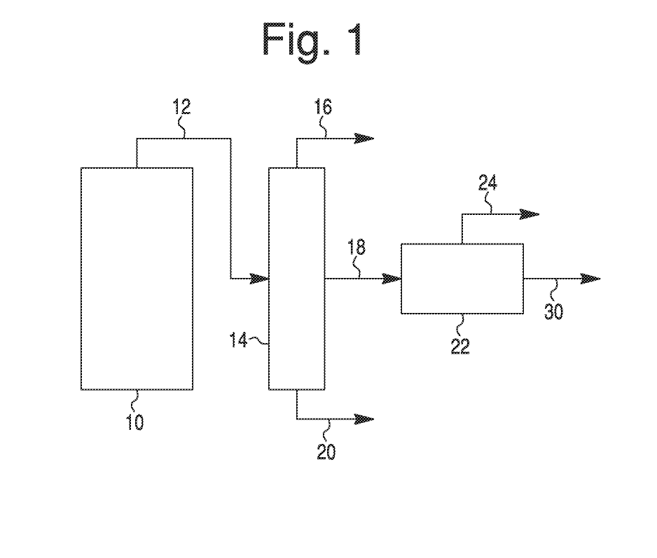

Reference is now made to the exemplary embodiment in FIG. 1. Suitable feed streams 30 for producing a desired xylene isomer, as described in detail below, are available from many sources. For example, a fluid catalytic cracking (FCC) unit 10, when run in high severity mode, produces a larger fraction of propylene and butylene than when run in standard operating modes. The FCC stream 12 discharged by the FCC unit 10 is fractionated in an FCC fractionator 14 to produce various fractions that are processed in different manners, such as a propylene stream 16, a C7-C10 stream 18, and a diesel stream 20. In some embodiments, the C7-C10 stream 18 includes about 60 mass percent aromatic compounds or more, and 60 mass percent aromatic compounds is an aromatic rich stream. Increased demand for propylene and butylene, combined with decreased demand for gasoline, provides an incentive to operate the FCC unit 10 in high severity mode to better match production with commercial demands. The propylene and butylene fractions are commercially valuable, and it is desirable to utilize the aromatics in the C7-C10 stream 18 as a co-product. The C7-C10 stream 18 can be hydrotreated in a hydrotreating unit 22 to remove sulfur in a sour gas stream 24, because sulfur can poison catalysts and lower the quality of the xylene products. The feed stream 30 exits the hydrotreating unit 22 prior to any reforming process, because no reforming process is used on the components of the feed stream 30, so the feed stream 30 is derived from the FCC unit 10 without reforming.

There are several other possible sources for the feed stream 30 that are rich in aromatic compounds but have not been reformed. For example, certain coal liquefaction processes produce hydrocarbon streams rich in aromatic compounds, and these hydrocarbon streams are suitable for use as the feed stream 30. Other possible sources include various petroleum refining, thermal or catalytic cracking of hydrocarbons, or petrochemical conversion processes.

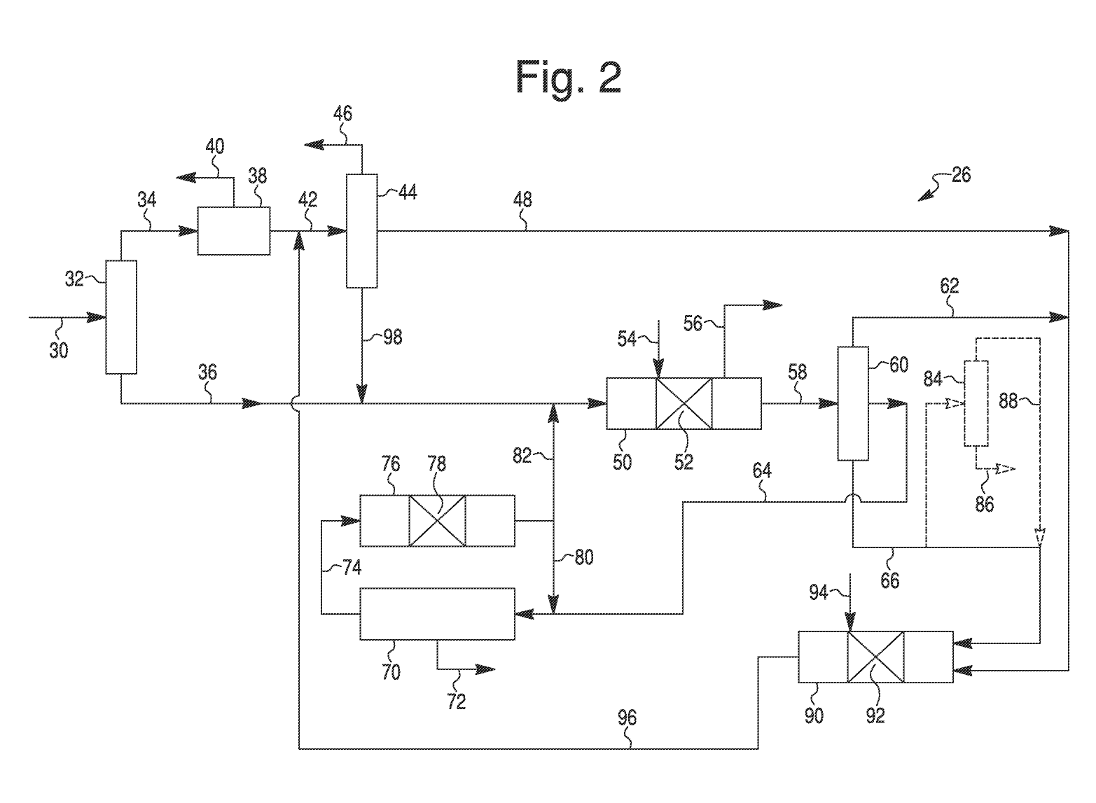

Reference is now made to an exemplary embodiment of a xylene isomer production system 26 illustrated in FIG. 2. The feed stream 30 is fed to a feed fractionator 32 and fractionated to produce a feed overhead stream 34 and a feed bottoms stream 36. The feed fractionator 32 can be operated from a pressure of about 5 kilo Pascals (KPa) to about 1,800 KPa, and a temperature from about 35 degrees centigrade (° C.) to about 360° C. The feed stream 30 includes mixed hydrocarbons with aromatic and non-aromatic compounds, and many of the hydrocarbons have from about 7 to about 10 carbons (C7-10). In some embodiments, the feed stream 30 has more than about 30 weight percent non-aromatic compounds, and in other embodiments the feed stream 30 has more than about 35 weight percent non-aromatic compounds. In still other embodiments the feed stream 30 includes non-aromatic compounds where the C8+ non-aromatic compounds are more than about 5 mass percent of the total non-aromatic compounds. Therefore, more than about 5 percent of the non-aromatic compounds in the feed stream 30 boil at a temperature greater than about 105° C. at 1 atmosphere of pressure, as described above. In yet another embodiment, the feed stream 30 has not been reformed. The feed fractionator 32 is operated such that the feed overhead stream 34 primarily includes C7− compounds, and the feed bottoms stream 36 primarily includes C8+ compounds.

The feed overhead stream 34 flows to an aromatics extraction zone 38 that produces a non-aromatics stream 40 and an aromatics stream 42. Any suitable process for separating high purity aromatics from non-aromatics may be employed in the aromatics extraction zone 38, including but not limited to liquid liquid extraction processes using sulfolane, crystallization extraction processes, or combinations of the two. The non-aromatics stream 40 is discharged from the xylene isomer production system 26, and can be used for other purposes. The aromatics stream 42, which primarily includes benzene and toluene, is routed to an aromatics fractionation zone 44.

The aromatics fractionation zone 44 includes one or more fractionation units that separate the aromatics stream 42 into a benzene stream 46 and a first light aromatics stream 48. The fractionation unit(s) in the aromatics fractionation zone 44 can be operated from a pressure of about 5 kilo Pascals (KPa) to about 1,800 KPa, and a temperature from about 35 degrees centigrade (° C.) to about 360° C. The benzene stream 46 primarily includes benzene, which is a valuable product, and the benzene stream 46 is discharged from the xylene isomer production system 26 and made available for other uses. The first light aromatics stream 48 primarily includes toluene, and is at least partially used within the xylene isomer production system 26 as further described below. In some embodiments, a portion of the first light aromatics stream 48 is optionally split off from the xylene isomer production system 26 (not illustrated).

Returning now to the feed fractionator 32, the feed bottoms stream 36 is transferred to a heavy aromatics conversion zone 50. The heavy aromatics conversion zone 50 includes a heavy aromatics conversion catalyst 52 that is tolerant of C9 aromatics. The heavy aromatics conversion catalyst 52 de-ethylates aromatic compounds with ethyl groups and changes the structure of some aromatic compounds, so ethyl benzene is converted to ethylene and benzene, toluene and methane, or a xylene. Other aromatic compounds with ethyl groups are also converted to benzene or aromatic compounds without ethyl groups. Benzene and aromatic compounds with methyl groups are generally more valuable than aromatic compounds with ethyl groups.

The heavy aromatics conversion zone 50 removes the remaining non-aromatic compounds from the feed bottoms stream 36 as well as de-ethylating the aromatic compounds. Smaller non-aromatic compounds, such C7−, are in the feed overhead stream 34, so larger non-aromatic compounds, such as C8+, are present in the feed bottoms stream 36. The heavy aromatics conversion catalyst 52 breaks non-aromatic compounds into smaller non-aromatic compounds, such as C4−. The smaller non-aromatic compounds are much more volatile than the C8+ aromatic compounds, and are vented off of the aromatics conversion zone 50 in a light gases stream 56. The smaller non-aromatic compounds in the light gases stream 56 are removed from the xylene isomer production system 26, and are available for other uses, so the remaining hydrocarbons are primarily aromatic compounds.

The heavy aromatics conversion zone 50 is operated at heavy aromatics conversion conditions in the presence of hydrogen, where the hydrogen is supplied by the heavy aromatics conversion hydrogen line 54. Suitable heavy aromatics conversion conditions include a temperature ranging from about 200° C. to about 600° C., or from about 300° C. to about 500° C. Suitable pressures are from about 100 KPa to about 5 mega Pascals (MPa) absolute, or from about 500 KPa to about 3 MPa absolute. The heavy aromatics conversion zone 50 contains a sufficient volume of heavy aromatics conversion catalyst 52 to provide a liquid hourly space velocity with respect to an intermediate stream (described below) from about 0.5 to about 50 hr⁻¹, or from about 0.5 to about 20 hr⁻¹. Hydrogen is provided from the heavy aromatics conversion hydrogen line 54 in a sufficient volume for a hydrogen/hydrocarbon mole ratio of about 0.5:1 to about 25:1. Other compounds may be present in the hydrogen, such as nitrogen, argon, or light hydrocarbons, without adverse effect.

The heavy aromatics conversion zone 50 is a single reactor in one exemplary embodiment, but in other embodiments it is two or more separate reactors with suitable means to ensure the desired isomerization temperature is maintained at the entrance to each reactor. The hydrocarbons are contacted with the heavy aromatics conversion catalyst 52 in any suitable manner, including upward flow, downward flow, or radial flow. The hydrocarbons may be in a liquid phase, a vapor phase, or a mixed liquid/vapor phase in the heavy aromatics conversion zone 50.

The heavy aromatics conversion catalyst 52 is an aromatics complex catalyst, and can include a zeolitic component, a metal component, and an inorganic oxide. Suitable zeolites include one or more of ATO, BEA, EUO, FAU, FER, MCM-22, MEL, MFI, MOR, MTT, MTW, NU-97 OFF, Omega, UZM-5, UZM-8, UZM-14, and TON, according to the Atlas of Zeolite Structure Types. The metal component includes one or more of the base noble metals in a proportion from about 0.01 weight percent to about 10 weight percent. Suitable metals include Rhenium (Re), Tin (Sn), Germanium (Ge), Lead (Pb), Cobalt (Co), Nickel (Ni), Indium (In), Gallium (Ga), Zinc (Zn), Uranium (U), Dysprosium (Dy), Thallium (Tl), Molybdenum (Mo), Ruthenium (Ru), Rhodium (Rh), Palladium (Pd), Osmium (Os), Iridium (Ir), and Platinum (Pt). The balance of the heavy aromatics conversion catalyst 52 can be an inorganic oxide binder, such as alumina. A variety of catalyst shapes can be used, such as spherical or cylinder shaped, but other shapes are also acceptable.

A de-ethylated aromatics stream 58 exits the heavy aromatics conversion zone 50, where the de-ethylated aromatics stream 58 primarily includes C10− aromatic compounds. Any non-aromatic compounds introduced to the heavy aromatics conversion zone 50 are converted to smaller non-aromatic compounds that are vented off in the light gases stream 56, as described above. The de-ethylated aromatics stream 58 feeds a heavy aromatics fractionator 60, which produces a second light aromatics stream 62, an intermediate aromatics stream 64, and a heavy aromatics stream 66. The second light aromatics stream 62 is primarily C7− aromatics, the intermediate aromatics stream 64 is primarily C8 aromatics, and the heavy aromatics stream 66 is primarily C9+ aromatics. The heavy aromatics fractionator 60 is one, two, or more fractionators in various embodiments, and suitable operating conditions include a temperature from about 35° C. to about 360° C. and a pressure from about 5 KPa to about 1,800 KPa.

The intermediate aromatics stream 64 includes the xylene compounds, and is fed to the isomer recovery process 70. The process employed to recover a particular desired isomer in the isomer recovery process 70 is not critical, and any effective recovery scheme known in the art may be used. For example, selective adsorption with a crystalline aluminosilicate adsorbent, crystallization processes, or combinations of the two can be used. The isomer recovery process 70 produces a desired isomer stream 72 and an isomer raffinate stream 74. The desired isomer stream 72 primarily includes one of the xylene isomers. In one embodiment, the para xylene is the isomer primarily present in the desired isomer stream 72, but in other embodiments ortho xylene or meta xylene is primarily present. The isomer raffinate stream 74 primarily includes the two xylene isomers that are not present in the desired isomer stream 72.

The isomer raffinate stream 74 flows to the isomerization zone 76, where the xylenes are isomerized to a ratio closer to the equilibrium ratio for xylene. One of the xylene isomers was removed in the isomer recovery process 70, and the removal of one isomer shifts the composition of the isomer raffinate stream 74 away from equilibrium. The isomer raffinate stream 74 primarily includes 2 of the 3 xylene isomers, so the third isomer is produced in the isomerization zone 76 to bring the mixture closer to an equilibrium ratio. The equilibrium ratio is about 20 to 25 percent ortho xylene, 20 to 30 percent para xylene, and 50 to 60 percent meta xylene at about 250° C., and this equilibrium ratio varies with temperature and other conditions.

The isomerization zone 76 includes an isomerization catalyst 78, and operates at suitable isomerization conditions. Suitable isomerization conditions include a temperature from about 100° C. to about 500°, or from about 200° C. to about 400° C., and a pressure from about 500 KPa to 5 MPa absolute. The isomerization unit includes a sufficient volume of isomerization catalyst 78 to provide a liquid hourly space velocity, with respect to the isomer raffinate stream 74, from about 0.5 to about 50 hr⁻¹, or from about 0.5 to about 20 hr⁻¹. Hydrogen may be present up to about 15 moles per mole of xylene, but in some embodiments hydrogen is essentially absent from the isomerization zone 76. In embodiments where hydrogen is essentially absent from the isomerization zone 76, no free hydrogen is added and residual dissolved hydrogen from prior processing is less than about 0.05 moles of hydrogen per mole of aromatic compound in the isomer raffinate stream 74. In other embodiments, hydrogen is present at less than about 0.01 moles of hydrogen per mole of aromatic compound in the isomer raffinate stream 74. The isomerization zone 76 may include one, two, or more reactors, where suitable means are employed to ensure a suitable isomerization temperature at the entrance to each reactor. The xylenes are contacted with the isomerization catalyst 78 in any suitable manner, including upward flow, downward flow, or radial flow.

The isomerization catalyst 78 includes a zeolitic aluminosilicate with a Si:Al₂ ratio greater than about 10, or greater than about 20 in some embodiments, and a pore diameter of about 5 to about 8 angstroms. Some examples of suitable zeolites include, but are not limited to, MFI, MEL, EUO, FER, MFS, MTT, MTW, TON, MOR, and FAU, and gallium may be present as a component of the crystal structure. In some embodiments, the Si:Ga₂ mole ratio is less than 500, or less than 100 in other embodiments. The proportion of zeolite in the catalyst is generally from about 1 to about 99 weight percent, or from about 25 to about 75 weight percent. In some embodiments, the isomerization catalyst 78 includes about 0.01 to about 2 weight percent of one or more of Ru, Rh, Pd, Os, Ir, and Pt, but in other embodiments the isomerization catalyst 78 is substantially absent of any metallic compound, where substantial absence is less than about 0.01 weight percent. The balance of the isomerization catalyst 78 is an inorganic oxide binder, such as alumina, and a wide variety of catalyst shapes can be used, including spherical or cylindrical.

An isomerized stream 80 exits the isomerization zone 76 and returns to the isomer recovery process 70, so the xylenes make a loop and repeatedly passes between the isomer recovery process 70 and the isomerization zone 76. The isomerized stream 80 includes more of the xylene isomer primarily present in the desired isomer stream 72 than in the isomer raffinate stream 74, so more of the desired xylene isomer is available for recovery. In this manner, the total amount of the desired xylene isomer recovered can exceed the equilibrium value of the desired xylene isomer. An isomerization purge stream 82 is also taken from the isomerization zone 76 to remove small concentrations of ethyl benzene and other lighter and heavier compounds produced in the isomerization zone 76 to prevent the build-up of these materials in the isomerization zone 76/isomer recovery process 70 loop. The isomerization purge stream 82 is fed into the heavy aromatics conversion zone 50, described above. The isomerization zone 76/isomer recovery process 70 loop operates without fractionating the xylenes, so far less energy is used than for other processes that do fractionate the xylenes in isomerization zone 76/isomer recovery process 70 loops.

As mentioned above, the heavy aromatics stream 66 primarily includes C9+ aromatics. The heavy aromatics stream 66 can optionally be fractionated in a pre-transalkylation fractionator 84 to produce a C10+ aromatics stream 86 and a C9 aromatics stream 88. The C10+ aromatics stream 86 primarily includes C10+ aromatics that are removed from the xylene isomer production system 26, and are available for other uses. The C9 aromatics stream 88 and/or the heavy aromatics stream 66 are then introduced to a transalkylation zone 90. The first light aromatics stream 48 and the second light aromatics stream 62, which are both primarily toluene, are also introduced to the transalkylation zone 90. The transalkylation zone 90 converts some of the C9+ aromatics from the heavy aromatics stream 66, preferably in the presence of toluene from the first and second light aromatics stream 48, 62, to C8 aromatic compounds. The transalkylation zone 90 further increases the yield of the desired xylene isomer by converting C9+ and C7 aromatics to C8 aromatics.

The transalkylation zone 90 includes a transalkylation catalyst 92, and the transalkylation zone 90 is operated in the presence of hydrogen supplied by the transalkylation hydrogen line 94 at suitable transalkylation conditions. Suitable transalkylation conditions include a temperature ranging from about 200° C. to about 600° C., for example from about 300° C. to about 500° C. Suitable pressures are from about 100 KPa to about 5 mega Pascals (MPa) absolute, for example from about 500 KPa to about 3 MPa. The transalkylation zone 90 contains a sufficient volume of transalkylation catalyst 92 to provide a liquid hourly space velocity with respect to a transalkylation stream 96 (described below) from about 0.5 to about 50 hr⁻¹, or from about 0.5 to about 20 hr⁻¹. Hydrogen is provided from the transalkylation hydrogen line 94 in a sufficient volume for a hydrogen/hydrocarbon mole ratio of about 0.5:1 to about 25:1. Other compounds may be present in the hydrogen, such as nitrogen, argon, or light hydrocarbons, with adverse effect.

The transalkylation zone 90 is a single reactor in one exemplary embodiment, but in other embodiments it is two or more separate reactors with suitable means to ensure the desired transalkylation temperature is maintained at the entrance to each reactor. The hydrocarbons are contacted with the transalkylation catalyst 92 in any suitable manner, including upward flow, downward flow, or radial flow. The hydrocarbons may be in a liquid phase, a vapor phase, or a mixed liquid/vapor phase in the transalkylation zone 90.

In exemplary embodiments, the transalkylation catalyst 92 includes a zeolitic component, a metal component, and an inorganic oxide. Suitable zeolites include one or more of ATO, BEA, EUO, FAU, FER, MCM-22, MEL, MFI, MOR, MTT, MTW, NU-97 OFF, Omega, mordenite, UZM-5, UZM-8, UZM-14, and TON, according to the Atlas of Zeolite Structure Types. The proportion of zeolite in the transalkylation catalyst 92 is from about 1 to about 99 weight percent, or from about 25 to about 75 weight percent. The metal component includes one or more of the base noble metals in a proportion from about 0.01 weight percent to about 10 weight percent. Suitable metals include Re, Sn, Ge, Pb, Co, Ni, In, Ga, Zn, U, Dy, Tl, Mo, Ru, Rh, Pd, Os, Ir, and Pt. The balance of the transalkylation catalyst 92 can be an inorganic oxide binder, such as alumina. A variety of catalyst shapes can be used, such as spherical or cylinder shaped, but other shapes are also possible.

A transalkylation stream 96 is produced by the transalkylation zone 90. The transalkylation stream primarily includes C7-10 aromatics, including many C8+ aromatics. The C8+ aromatics from the transalkylation stream 96 are fed into the heavy aromatic conversion zone 50 and contact the heavy aromatic conversion catalyst 52. Several different embodiments can be used to transfer the C8+ aromatics from the transalkylation stream 96 into the heavy aromatic conversion zone 50. In one exemplary embodiment, the transalkylation stream 96 is fed into the aromatics fractionation zone 44, and an aromatics fractionation bottoms stream 98 is fed into the heavy aromatic conversion zone 50. The aromatics fractionation zone 44 is operated so the aromatics fractionation bottoms stream 98 includes C8+ aromatics, which are introduced to the aromatics fractionation zone 44 by the transalkylation stream 96. In other embodiments (not shown), the transalkylation stream 96 is directly fed into the heavy aromatics conversion zone 50, and thereby transfers the C8+ aromatics as well as the C7− aromatics and any other compounds present. In yet another embodiment (not shown), a separate fractionation column is used to separate the components of the transalkylation stream 96 and feed the C8+ aromatics to the heavy aromatic conversion zone 50.

Many different embodiments are possible, so it should be appreciated that a vast number of variations exist. It should also be appreciated that the embodiment or embodiments illustrated are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described without departing from the scope as set forth in the appended claims.

1. A method of producing xylenes, the method comprising the steps of:
fractionating a feed stream in a feed fractionator to produce a feed bottoms stream and a feed overhead stream, wherein the feed bottoms stream comprises aromatic compounds with 8 carbons or more and the feed overhead stream comprises aromatic compounds with 7 carbons or less, wherein the feed stream comprises non-aromatic compounds and the aromatic compounds, and wherein more than 5 weight percent of the non-aromatic compounds in the feed stream have a boiling point above 105 degrees centigrade at one atmosphere of pressure;
de-ethylating the feed bottoms stream in a heavy aromatics conversion zone to produce a de-ethylated aromatics stream, wherein the heavy aromatics conversion zone produces a light gases stream from the non-aromatic compounds;
fractionating the de-ethylated aromatics stream in a heavy aromatics fractionator to produce a heavy aromatics stream and an intermediate aromatics stream, wherein the heavy aromatics stream comprises the aromatic compounds with 9 carbons or more and the intermediate aromatics stream comprise the aromatic compounds with 8 carbons;
recovering a desired isomer stream from the intermediate aromatics stream and an isomerized stream in an isomer recovery process, wherein the isomer recovery process produces an isomer raffinate stream, and wherein the desired isomer stream comprises a xylene isomer; and
isomerizing the isomer raffinate stream in an isomerization zone to produce the isomerized stream.

2. The method of claim 1 further comprising:
subjecting the feed overhead stream to an aromatics extraction zone that produces a non-aromatics stream and an aromatics stream;
fractionating the aromatics stream in an aromatics fractionation zone to produce a benzene stream and a first light aromatics stream, wherein the first light aromatics stream comprises toluene;

3. The method of claim 2 further comprising:
transalkylating the heavy aromatics stream with the first light aromatics stream to produce a transalkylation stream comprising the aromatic compounds with 8 carbons or more.

4. The method of claim 3 wherein transalkylating the heavy aromatics stream further comprises fractionating the transalkylation stream in the aromatics fractionation zone to produce an aromatics fractionation bottoms stream; and wherein
de-ethylating the feed bottoms stream in the heavy aromatics conversion zone further comprises de-ethylating the aromatics fractionation bottoms stream in the heavy aromatics conversion zone.

5. The method of claim 1 wherein isomerizing the isomer raffinate stream further comprises isomerizing the isomer raffinate stream at isomerization conditions comprising less than about 0.05 moles of hydrogen per mole of the aromatic compounds in the isomer raffinate stream.

6. The method of claim 5 wherein isomerizing the isomer raffinate stream further comprises isomerizing the isomer raffinate stream with an isomerization catalyst comprising from about 10 to about 99 weight percent of a zeolitic aluminosilicate and an inorganic-oxide binder and has a substantial absence of Ruthenium, Rhodium, Palladium, Osmium, Iridium, and Platinum.

7. The method of claim 1 wherein fractionating the feed stream in the feed fractionator further comprises fractionating the feed stream in the feed fractionator wherein the feed stream is introduced to the feed fractionator prior to any reforming process.

8. The method of claim 1 wherein fractionating the feed stream in the feed fractionator further comprises fractioning the feed stream in the feed fractionator wherein the feed stream is derived from a fluid catalytic cracking unit.

9. A method of producing xylenes, the method comprising the steps of:
fractionating a feed stream in a feed fractionator to produce a feed bottoms stream comprising aromatic compounds with 8 carbon atoms or more and a feed overhead stream comprising the aromatic compounds with 7 carbon atoms or less, and wherein the feed stream comprises more than 30 weight percent non-aromatic compounds;
separating compounds in the feed overhead stream to produce a non-aromatics stream and a first light aromatics stream, wherein the first light aromatics stream comprises the aromatic compounds with 7 carbons;
contacting the feed bottoms stream with a heavy aromatics conversion catalyst at heavy aromatics conversion conditions to obtain a de-ethylated aromatics stream and a light gases stream;
fractionating the de-ethylated aromatics stream to produce a heavy aromatics stream comprising the aromatic compounds with 9 carbons or more and an intermediate aromatics stream comprising the aromatic compounds with 8 carbons;
subjecting the intermediate aromatics stream and an isomerized stream to an isomer recovery process to produce a desired isomer stream and an isomer raffinate stream, wherein the desired isomer stream comprises a xylene isomer;
contacting the isomer raffinate stream with an isomerization catalyst at isomerization conditions to produce the isomerized stream; and
contacting the heavy aromatics stream and the first light aromatics stream with a transalkylation catalyst at transalkylation conditions to obtain a transalkylation stream comprising the aromatic compounds with 8 carbons or more.

10. The method of claim 9 wherein contacting the isomer raffinate stream with the isomerization catalyst at the isomerization conditions further comprises contacting the isomer raffinate stream with the isomerization catalyst at the isomerization conditions, wherein the isomerization conditions comprise less than about 0.05 moles of hydrogen per mole of the aromatic compounds in the isomer raffinate stream;

11. The method of claim 9 wherein contacting the heavy aromatics stream and the first light aromatics stream with the transalkylation catalyst further comprises fractionating the transalkylation stream in an aromatics fractionation zone to produce an aromatics fractionation bottoms stream comprising the aromatic compounds with 8 carbons or more; and wherein
contacting the feed bottoms stream with the heavy aromatics conversion catalyst further comprises contacting the aromatics fractionation bottoms stream with the heavy aromatics conversion catalyst.

12. The method of claim 9 wherein contacting the isomer raffinate stream with the isomerization catalyst further comprises contacting the isomer raffinate stream with the isomerization catalyst wherein the isomerization catalyst comprises from about 10 to 99 weight percent of a zeolitic aluminosilicate and an inorganic-oxide binder and has a substantial absence of Ruthenium, Rhodium, Palladium, Osmium, Iridium, and Platinum.

13. The method of claim 9 wherein fractionating the feed stream in the feed fractionator further comprises fractionating the feed stream in the feed fractionator wherein the feed stream is introduced to the feed fractionator prior to any reforming process.

14. The method of claim 9 wherein fractionating the feed stream in the feed fractionator further comprises fractioning the feed stream in the feed fractionator wherein the feed stream is derived from a fluid catalytic cracking unit.

15. A method of producing xylenes, the method comprising the steps of:
fractionating a feed stream in a feed fractionator to produce a feed bottoms stream and a feed overhead stream, wherein the feed overhead stream comprises aromatic compounds with 7 carbons or less, the feed bottoms stream comprises the aromatic compounds with 8 carbons or more, and wherein the feed stream is introduced to the feed fractionator prior to any reforming process;
subjecting the feed overhead stream to an aromatics extraction zone that produces a non-aromatics stream and an aromatics stream;
fractionating the aromatics stream in an aromatics fractionation zone to produce a benzene stream and a first light aromatics stream, wherein the first light aromatics stream comprises the aromatic compounds with 7 carbons;
de-ethylating the feed bottoms stream in a heavy aromatics conversion zone to produce a de-ethylated aromatics stream, wherein the heavy aromatics conversion zone produces light gases from non-aromatic compounds;
fractionating the de-ethylated aromatics stream in a heavy aromatics fractionator to produce a heavy aromatics stream and an intermediate aromatics stream, wherein the heavy aromatics stream comprises the aromatic compounds with 9 carbons or more and the intermediate aromatics stream comprises the aromatic compounds with 8 carbons;
recovering a desired isomer stream from the intermediate aromatics stream and an isomerized stream in an isomer recovery process, wherein the isomer recovery process produces an isomer raffinate stream, and wherein the desired isomer stream comprises a xylene isomer;
isomerizing the isomer raffinate stream in an isomerization zone to produce the isomerized stream; and
transalkylating the heavy aromatics stream with the first light aromatics stream to produce a transalkylation stream comprising the aromatic compounds with 8 carbons or more.

16. The method of claim 15 wherein isomerizing the isomer raffinate stream further comprises isomerizing the isomer raffinate stream in the isomerization zone at isomerization conditions comprising less than about 0.05 moles of hydrogen per mole of the aromatic compounds in the isomer raffinate stream.

17. The method of claim 15 wherein isomerizing the isomer raffinate stream further comprises isomerizing the isomer raffinate stream with an isomerization catalyst comprising from about 10 to about 99 weight percent of a zeolitic aluminosilicate and an inorganic-oxide binder and has a substantial absence of Ruthenium, Rhodium, Palladium, Osmium, Iridium, and Platinum.

18. The method of claim 15 further comprising
fractionating the transalkylation stream in the aromatics fractionation zone to produce an aromatics fractionation bottoms stream comprising the aromatic compounds with 8 carbons or more; and wherein
de-ethylating the feed bottoms stream in the heavy aromatics conversion zone further comprises de-ethylating the aromatics fractionation bottoms stream in the heavy aromatics conversion zone.

19. The method of claim 15 wherein fractionating the de-ethylated aromatics stream further comprises producing a second light aromatics stream; and wherein
transalkylating the heavy aromatics stream with the first light aromatics stream further comprises transalkylating the heavy aromatics stream with the first light aromatics stream and with the second light aromatics stream.

20. The method of claim 15 wherein fractionating the feed stream further comprises fractionating the feed stream wherein the feed stream comprises more than 30 weight percent of the non-aromatic compounds.
